(19) United States
(12) Patent Application Publication
(10) Pub. No.: US 2017/0119995 A1
(43) Pub. Date: May 4, 2017

(54) SLEEP CONTROL SYSTEM

(71) Applicant: SHARP KABUSHIKI KAISHA, Sakai City, Osaka (JP)

(72) Inventors: Tomohiro KOYANAGI, Sakai City (JP); Yukio YAMASAKI, Sakai City (JP)

(21) Appl. No.: 15/318,374

(22) PCT Filed: Jun. 1, 2015
(86) PCT No.: PCT/JP2015/065811

§ 371 Date: Dec. 13, 2016

(30) Foreign Application Priority Data
Jun. 19, 2014 (JP) 2014-125995
Dec. 8, 2014 (JP) 2014-247514

(51) Int. Cl. A61M 21/02; A61F 7/08

(52) U.S. Cl. CPC A61M 21/02; A61F 7/08; A61F 2007/0086

(57) ABSTRACT

A sleep control system in accordance with an embodiment of the present invention includes: a heating section being provided in contact with a human body and heating the human body; and a control section for controlling a heating operation carried out by the heating section, the heating section carrying out heating at a temperature in a range of 36° C. to 41° C. in a period from time for starting heating until a scheduled awakening time, the time for staring heating being in a period of 90 minutes to 150 minutes prior to the scheduled awakening time, and the heating section carrying out no heating at a temperature higher than 33° C. in a period from bedtime until the time for starting heating or in a period after elapse of a given time from the bedtime and until the time for starting heating.

### TECHNICAL FIELD

The present invention relates to a sleep control system, in particular, to a sleep control system that leads to pleasant awakening.

### BACKGROUND ART

People nowadays tend to sleep for shorter hours, and at the same time, get up by necessity at a substantially constant time in the mornings so as to participate in social activities. Many people substantially forcibly get up by using an alarm clock or the like. In such people, the feeling of lack of sleep, sleep inertia, decrease in activity right after awakening, and the like occur. In order to remedy such problems, the following is disclosed as a sleep control heating apparatus.

FIG. 14 is a view schematically illustrating a configuration of a sleep control electric blanket disclosed in Patent Literature 1. A main blanket body 101 heats an upper body of a human by an upper heater 102 and a lower body of the human by a lower heater 103. Further, the main blanket body 101 detects a heating temperature for the upper body by an upper temperature sensor 104 and a heating temperature for the lower body by a lower temperature sensor 105. The respective heating temperatures of the upper heater 102 and the lower heater 103 are adjusted by a controller 106, on the basis of detection results of the upper temperature sensor 104 and the lower temperature sensor 105.

FIG. 15 is a time chart showing respective changes in the heating temperatures of the upper body and the lower body in the sleep control electric blanket disclosed in Patent Literature 1. In an awakening mode for a period from 45 minutes prior to awakening until the time of awakening, the heating temperature for the upper body is controlled so as to be in a range of 37° C. to 38° C. while the heating temperature of the lower body is controlled so as to be in a range of 31° C. to 33° C. This suppresses heat release from feet, while the temperature of a body trunk increases. This gradually increases an arousal level of a body of a human in sleep, so that awakening from sleep occurs in a state in which non rapid-eye movement sleep (NREM sleep) has shifted to rapid-eye movement sleep (REM sleep).

### CITATION LIST

### Patent Literature

[Patent Literature 1] Japanese Patent Publication No. 3960772 (Publication Date: Aug. 15, 2007)

### SUMMARY OF INVENTION

### Technical Problem

The sleep control electric blanket disclosed in Patent Literature 1 starts increasing body temperature 45 minutes prior to awakening. Accordingly, in view of approximately 90-minute cycles of REM and NREM sleep, the body temperature may not be able to be increased in a period of REM sleep. Such increase in temperature is not sufficient for leading to a pleasant awakening state. A sleep rhythm of a human is unique to a human body and cannot be easily changed. For example, in a case where REM sleep is exhibited 55 minutes prior to a scheduled awakening time, the REM sleep does not continue for up to approximately 55 minutes until awakening even when heating is started 10 minutes after exhibition of the REM sleep, that is, from 45 minutes prior to awakening. In other words, exhibition of REM sleep by heating for a short period of time largely depends on chance. Therefore, the sleep control electric blanket disclosed in Patent Literature 1 is not sufficient for leading to a pleasant awakening state.

An embodiment of the present invention is attained in view of the above problems. An object of an embodiment of the present invention is to provide a sleep control system capable of leading to a pleasant awakening state by increasing body temperature for an appropriate period of time.

### Solution to Problem

A sleep control system in accordance with an embodiment of the present invention includes: a heating section being provided in contact with a human body and heating the human body; and a control section for controlling a heating operation carried out by the heating section, the heating section carrying out heating at a temperature in a range of 36° C. to 41° C. in a period from time for starting heating until a scheduled awakening time, the time for staring heating being in a period of 90 minutes to 150 minutes prior to the scheduled awakening time, and the heating section carrying out no heating at a temperature higher than 33° C. in a period from bedtime until the time for starting heating or in a period after elapse of a given time from the bedtime and until the time for starting heating.

A sleep control system in accordance with an embodiment of the present invention includes: a heating section being provided in contact with a human body and heating the human body; and a control section for controlling a heating operation carried out by the heating section, the heating section not only carrying out heating at a temperature in a range of 36° C. to 41° C. in a period from time for starting heating until 0 minute to 30 minutes prior to a scheduled awakening time, but also carrying out heating at a temperature in a range of not lower than 31° C. and lower than 36° C. in a period of 0 minute to 30 minutes prior to the scheduled awakening time, the time for starting heating being in a period of 90 minutes to 150 minutes prior to the scheduled awakening time, and the heating section carrying out no heating at a temperature higher than 33° C. in a period from bedtime until the time for starting heating or in a period after elapse of a given time from the bedtime and until the time for starting heating.

An embodiment of the present invention may be arranged such that the heating section carries out no heating in the period from bedtime until the time for starting heating or in a period after elapse of a given time from the bedtime and until the time for starting heating.

An embodiment of the present invention may be arranged such that the heating section is provided below the human body.

An embodiment of the present invention may be arranged such that the heating section is formed so as to have a size corresponding to an upper body from shoulder tips to buttocks of the human body.

An embodiment of the present invention may be arranged to further include: a vibration sensor for detecting vibration of the human body, the control section setting a heating duration time and a heating temperature for carrying out heating by the heating section on the basis of information from the vibration sensor.

An embodiment of the present invention may be arranged to further includes: an audio device for outputting a sound, the control section causing a sound to be outputted from the audio device at the scheduled awakening time, the sound serving as an alarm for the scheduled awakening time.

### Advantageous Effects of Invention

A sleep control system in accordance with an embodiment of the present invention is capable of leading to a pleasant awakening state by increasing a body temperature for an appropriate period of time.

### BRIEF DESCRIPTION OF DRAWINGS

FIG. 1 is a perspective view schematically illustrating a sleep control system in accordance with Embodiment 1.

FIG. 2 is a block diagram illustrating a configuration of a control section in accordance with Embodiment 1.

FIG. 3 is a flowchart for describing an operation of the sleep control system in accordance with Embodiment 1.

FIG. 4 is a chart showing respective changes in human body temperature and in heating temperature of a heating section in accordance with Embodiment 1.

FIG. 5 is a graph for describing an effect of the sleep control system in accordance with Embodiment 1.

FIG. 6 is a perspective view schematically illustrating a sleep control system in accordance with Embodiment 2.

FIG. 7 is a block diagram illustrating a configuration of a control section in accordance with Embodiment 2.

FIG. 8 is a flowchart for describing an operation of a sleep control system in accordance with Embodiment 2.

FIG. 9 is a block diagram illustrating a configuration of a control section in accordance with Embodiment 3.

FIG. 10 is a flowchart for describing an operation of a sleep control system in accordance with Embodiment 3.

FIG. 11 is a chart showing a change in heating temperature of a heating section in accordance with Embodiment 4.

FIG. 12 is a chart showing a change in heating temperature of a heating section in accordance with Embodiment 5.

FIG. 13 is a chart showing a change in heating temperature of a heating section in accordance with Embodiment 6.

FIG. 14 is a view schematically illustrating a sleep control electric blanket in accordance with Patent Literature 1.

FIG. 15 is a time chart showing respective changes in heating temperatures of the upper body and the lower body, which changes are obtained by the sleep control electric blanket in accordance with Patent Literature 1.

### DESCRIPTION OF EMBODIMENTS

### Embodiment 1

The following discusses a sleep control system 1 in accordance with Embodiment 1, with reference to drawings.

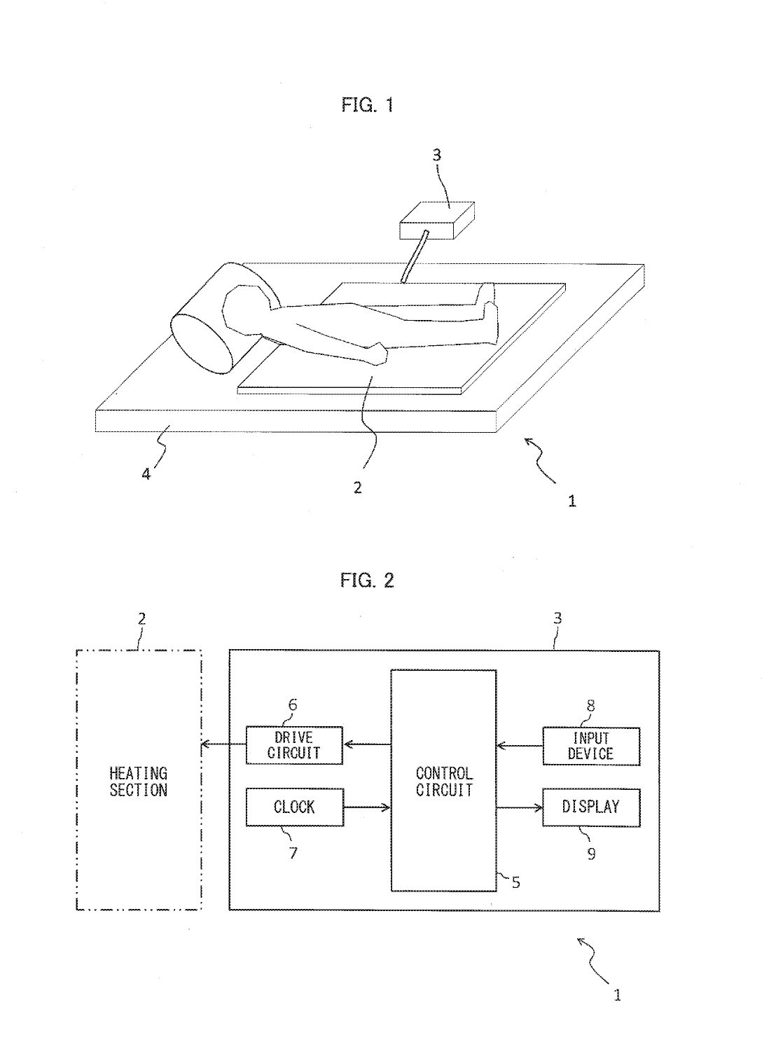

FIG. 1 is a perspective view schematically illustrating the sleep control system 1 of Embodiment 1. The sleep control system 1 of Embodiment 1 includes a heating section 2 for heating a human body, and a control section 3 for controlling a heating operation of the heating section 2.

The heating section 2 is provided on or above a mattress 4 and below a human body. The heating section 2 is provided in contact with the human body and arranged to generate heat by use of nichrome wire, so that the human body is heated. Further, in Embodiment 1, the heating section 2 is formed so as to have a size that allows for heating an area from shoulder tips to feet of the human body.

As one way for heating a human body, there is a method in which air temperature is increased by use of an air conditioner or the like. However, an increase in air temperature during sleep causes a person to feel discomfort, and consequently leads to arousal during sleep. This deteriorates the quality of sleep. It is thus not suitable to use an air conditioner or the like as the heating section 2. In Embodiment 1, the heating section 2 is in contact with a human body and heats the human body. Note that a state in which the heating section 2 is in contact with a human body encompasses not only a state in which a human body and the heating section 2 are put in direct contact with each other but also (i) a state in which clothes, sheets, and/or the like intervene between the human body and the heating section 2 and (ii) a state in which the heating section 2 is embedded in a mattress and accordingly padding or the like intervenes between the human body and the heating section 2. In other words, a state of being in contact includes a state in which there is an intervening material when viewed schematically.

FIG. 2 is a block diagram illustrating a configuration of the control section 3 used in Embodiment 1. The control section 3 includes a control circuit 5 including a CPU, a drive circuit 6 for heating the heating section 2 on the basis of information from the control circuit 5, a clock 7 for real-time measurement, an input device 8 for allowing a user to input a scheduled awakening time and/or the like, and a display 9 for displaying set conditions and/or the like. The control section 3 controls a heating temperature and an operation duration time of the heating section 2, in accordance with the scheduled awakening time. Note that the control circuit 5 contains a memory (not illustrated) for storing set conditions such as a scheduled awakening time.

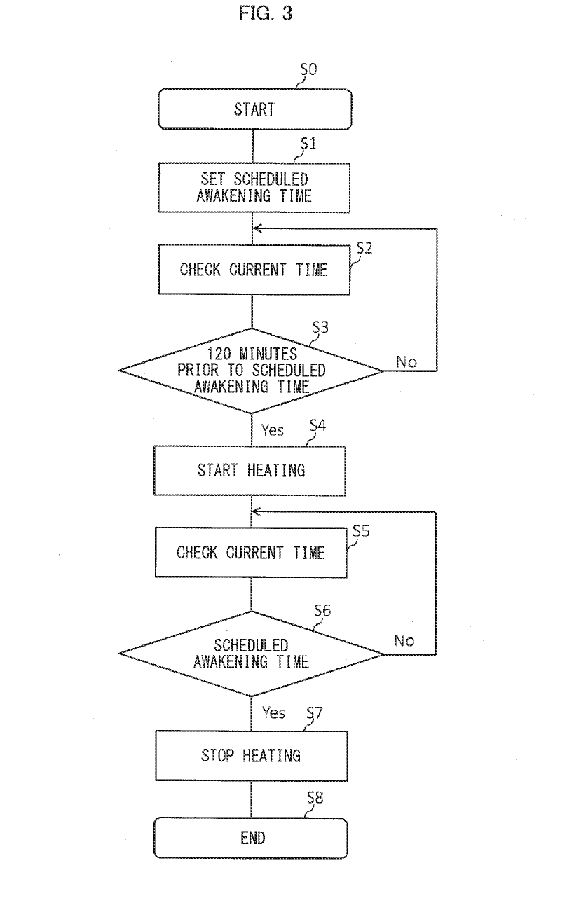

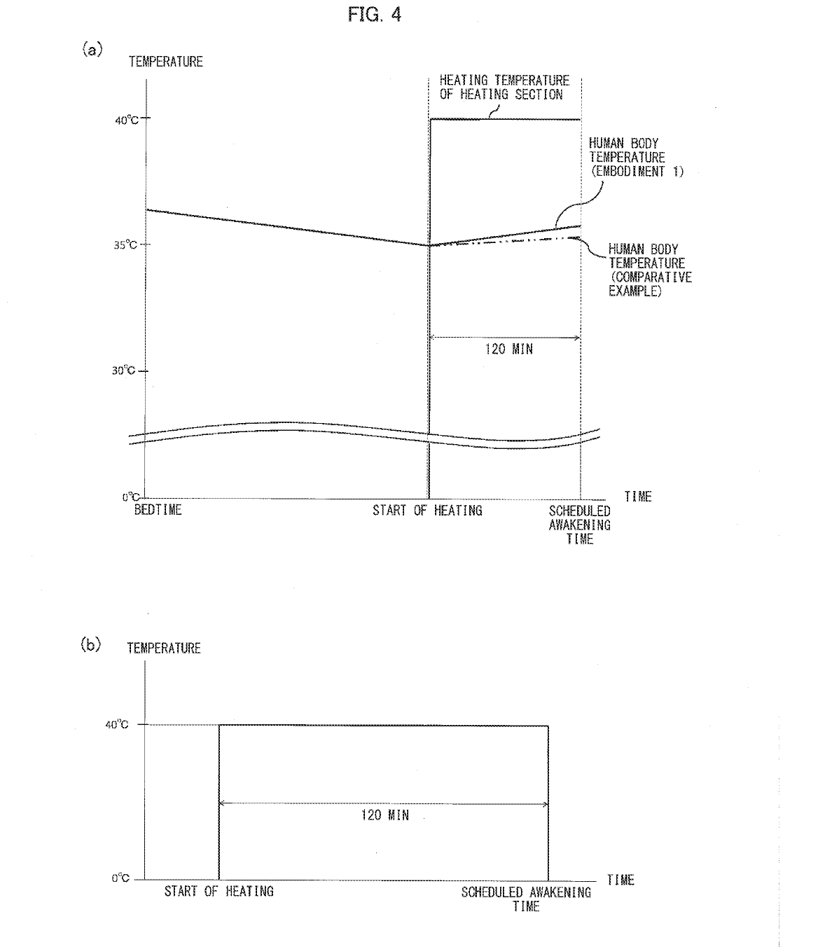

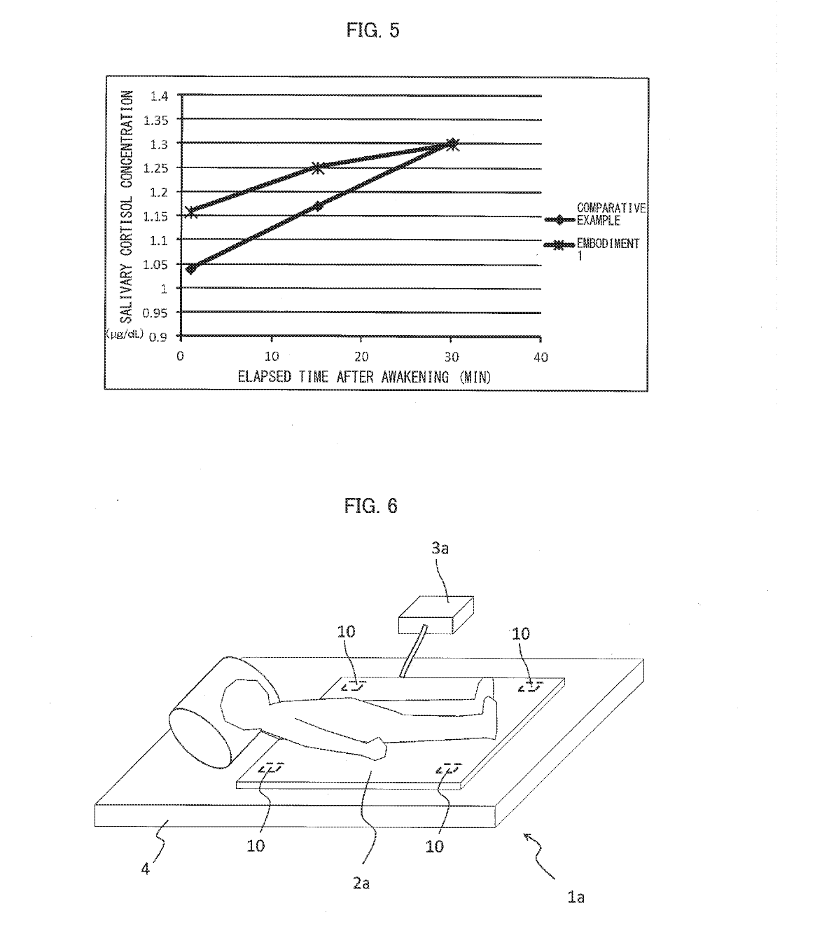

FIG. 3 is a flowchart for describing an operation of the sleep control system 1 of Embodiment 1. FIG. 4 is a chart showing respective changes in human body temperature and in heating temperature of the heating section 2 used in Embodiment 1. (a) of FIG. 4 shows respective changes in human body temperature and in heating temperature of the heating section 2. (b) of FIG. 4 shows a change in heating temperature of the heating section 2, from a start of heating to a scheduled awakening time. FIG. 5 is a graph for describing an effect of the sleep control system 1 used in Embodiment 1.

As shown in FIG. 3, in Step S0, a user turns on the sleep control system 1 at bedtime, so that the sleep control system 1 starts an operation.

In Step S1, the user inputs a scheduled awakening time via the input device 8, so that the scheduled awakening time is set in the control circuit 5.

In Step S2, the control circuit 5 checks the current time by the clock 7.

In Step S3, the control circuit 5 determines whether the current time is 120 minutes prior to the scheduled awakening time. Then, the sleep control system 1 shifts to Step S4 in a case where a determination result in Step 3 is Yes, whereas in a case where the determination result in Step 3 is No, the sleep control system 1 returns back to Step S2.

In Step S4, the heating section 2 starts heating in accordance with a signal generated by the drive circuit 6. Note that in Embodiment 1, the heating temperature of the heating section 2 is set at 40° C.

In Step S5, the control circuit 5 checks the current time by the clock 7.

In the Step S6, the control circuit 5 determines whether the current time is the scheduled awakening time. Then, the sleep control system 1 shifts to Step S7 in a case where a determination result in Step 6 is Yes, whereas in a case where the determination result in Step 5 is No, the sleep control system 1 returns back to Step S6.

In Step S7, the heating section 2 stops heating in accordance with a signal generated by the drive circuit 6.

In Step S8, the user turns off the sleep control system 1 after awakening, so that the sleep control system 1 ends the operation.

In Embodiment 1, according to Steps S3 and Step S4, the heating section 2 carries out heating at 40° C. from 120 minutes prior to the scheduled awakening time. The following discusses a reason why heating is carried out in such a way.

(a) of FIG. 4 shows, as a comparative example, a case where the heating section 2 does not carry out heating. A human body temperature is known to have a circadian rhythm. It is also known that as shown in the comparative example, according to the circadian rhythm, the human body temperature starts decreasing from before bedtime and further decreases by approximately 1° C. to 1.5° C. over several hours before awakening, and then increases. Note that in a case where such decrease of the human body temperature is inhibited, for example, the number of arousals during sleep increases and sleep efficiently deteriorates. This consequently causes a trouble in sleeping.

It is also known that during sleep, REM sleep that is shallow sleep and NREM sleep that is deep sleep are repeated in approximately 90-minute cycles. The length of a period of REM sleep in one cycle is considered to be approximately 10 minutes to 30 minutes, while the length of a period of NREM sleep in one cycle is considered to be approximately 60 minutes to 80 minutes. During a period of REM sleep, autonomic nerve is activated and blood flow is better than during a period of NREM sleep.

REM sleep is exhibited in 90-minute cycles and the heating section 2 takes 30 minutes to increase and stabilize in temperature. On this account, in Embodiment 1, the heating section 2 carries out heating in a period from 120 minutes prior to a scheduled awakening time until the scheduled awakening time. Accordingly, in Embodiment 1, heating is carried out at a temperature higher than a body temperature in a last period of REM sleep prior to the scheduled awakening time. In this period of REM sleep, heat is distributed every part of a human body, because autonomic nerve is activated and blood flow is good.

As shown in (a) of FIG. 4, in Embodiment 1, a body temperature starts decreasing from before bedtime and further decreases over several hours before awakening, as in the comparative example. However, in Embodiment 1, the body temperature is higher than that in the comparative example, from 120 minutes prior to a scheduled awakening time until the scheduled awakening time. Since the body temperature at the time of awakening can be higher in Embodiment 1 than in the comparative example, an arousal level after awakening is higher in Embodiment 1.

FIG. 5 shows a result of measurement of a salivary cortisol concentration by EIA method, as evaluation of an effect of the sleep control system 1. The salivary cortisol concentration serves as an index of an arousal level after awakening. In postdormitum, a person exhibits a phenomenon in which an amount of secretion of cortisol is increased. This phenomenon is a preparation for awakening, and the cortisol here is corticosteroid. Cortisol acts to induce energy production by glycolysis.

As shown in FIG. 5, in Embodiment 1, the salivary cortisol concentration at the time of awakening is higher in Embodiment 1 than the comparative example. Accordingly, a person at the time of awakening is in a state in which more energy can be utilized for activities after awakening in Embodiment 1. In other words, it is clear that the arousal level after awakening is higher in Embodiment 1, and this leads to a pleasant awakening state.

Note that though Embodiment 1 assumes that the length of a cycle of REM and NREM sleep is approximately 90 minutes and accordingly, the heating section 2 starts heating from 120 minutes prior to a scheduled awakening time in Embodiment 1, an embodiment of the present invention is not limited to this configuration. The length of a cycle of REM and NREM sleep depends on each individual. Accordingly, on assumption that cycles of various individuals are to be dealt with, the length of a cycle of REM and NREM sleep needs to be set so as to range from 60 minutes to 120 minutes. Then, the time for starting heating by the heating section 2 is accordingly set to range from 90 minutes to 150 minutes prior to a scheduled awakening time. Within such a range, the time for starting heating should be set in consideration of each individual.

In the heating section 2 here, in a case where heating is started from a time point that is earlier than 90 minutes to 150 minutes prior to a scheduled awakening time, a body temperature does not appropriately decrease. This leads to for example, increase in the number of arousals during sleep and deteriorates sleep efficiency. This consequently causes a trouble in sleeping. Meanwhile, in a case where heating is started from a time point that is later than 90 minutes prior to a scheduled awakening time, REM sleep may not be exhibited during heating by the heating section 2. In other words, in order to lead to a pleasant awakening state, the heating section 2 needs to start heating from 90 minutes to 150 minutes prior to a scheduled awakening time.

Though in Embodiment 1, the heating section 2 carries out heating at 40° C., an embodiment of the present invention is not limited to this configuration. In consideration of an average human body temperature and prevention of low temperature burn, the heating section 2 should carry out heating at a temperature in a range of 36° C. to 41° C.

Further, though in Embodiment 1, the heating section 2 is formed so as to have a size corresponding to an area from shoulder tips to feet of a human body, an embodiment of the present invention is not limited to this configuration. The heating section 2 only needs to cover an upper body of a human body from shoulder tips to buttocks, and does not need to cover feet. Meanwhile, the heating section may be extended so as to reach a head region. However, heating the head region may cause discomfort, and tends to cause arousal during sleep. Accordingly, heating the head region consequently deteriorates the quality of sleep.

Further, though in Embodiment 1, the heating section 2 is provided below a human body, an embodiment of the present invention is not limited to this configuration. In Embodiment 1, the heating section 2 only needs to be in contact with a human body and heat the human body. The position of the heating section 2 does not necessarily need to be limited to a position below a human body, but may be provided, for example, on or above a human body. In such a case, the heating section 2 may be provided as a part of a comforter. Alternatively, the heating section 2 may be provided on or above a comforter. That is, the heating section 2 may be provided such that the comforter is sandwiched between a human body and the heating section 2. Even in a case where the comforter or the like is sandwiched between the human body and the heating section 2, the heating section 2 is still understood to be in contact with a human body and heat the human body, via a comforter or the like.

However, in a case where the heating section 2 is provided on or above a human body, the human body is positioned between the heating section 2 and the mattress 4. This makes heat release difficult according to the heat convection principle. Accordingly, this may result in increase in temperature surrounding the human body, and cause discomfort. Further, in a case where the heating section 2 is provide such that a comforter or the like is sandwiched between a human body and the heating section 2, transfer of heat to the human body becomes difficult. This may cause an embodiment of the present invention to yield an insufficient effect. Furthermore, though in Embodiment 1, the heating section 2 is provided on or above the mattress 4, an embodiment of the present invention is not limited to this configuration. For example, the heating section 2 may be provided inside the mattress 4.

Moreover, though in Embodiment 1, the heating section 2 generates heat by heating wire in which nichrome wire is used, an embodiment of the present invention is not limited to this configuration. For example, heating may be carried out by use of far-infrared radiation.

Further, in Embodiment 1, though the heating section 2 does not carry out heating in a period from around bedtime until 90 minutes to 150 minutes prior to a scheduled awakening time, the heating section 2 may carry out heating at a temperature of not higher than 33° C. The temperature of not higher than 33° C. is unlikely to inhibit decrease in body temperature during sleep, and accordingly, is unlikely to cause a problem of deterioration in sleep efficiency. Alternatively, the heating section 2 may be arranged to carry out heating, for example, at not lower than 33° C. for 5 minutes right after bedtime, and thereafter, not to carry out heating or carry out heating at not higher than 33° C. until 90 minutes to 150 minutes prior to a scheduled awakening time. However, it is more preferable to carry out heating in advance of bedtime to a degree that prevents bedclothes from being felt cold, and not to carry out heating during hours of sleeping. This is because arousal during sleep is not increased in such a case.

In addition, though in Embodiment 1, a user having awakened turns off the sleep control system 1 and ends an operation of the sleep control system 1, an embodiment of the present invention is not limited to this configuration. The sleep control system 1 may be turned off automatically.

### Embodiment 2

The following discusses a sleep control system 1a in accordance with Embodiment 2, with reference to drawings. Embodiment 2 is different from Embodiment 1 in that the sleep control system 1a is provided with a vibration sensor 10. The description of Embodiment 2 puts the focus on parts different from Embodiment 1.

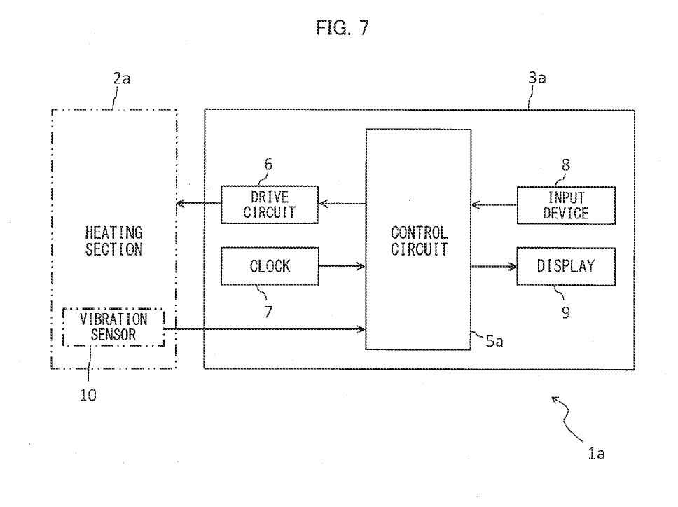

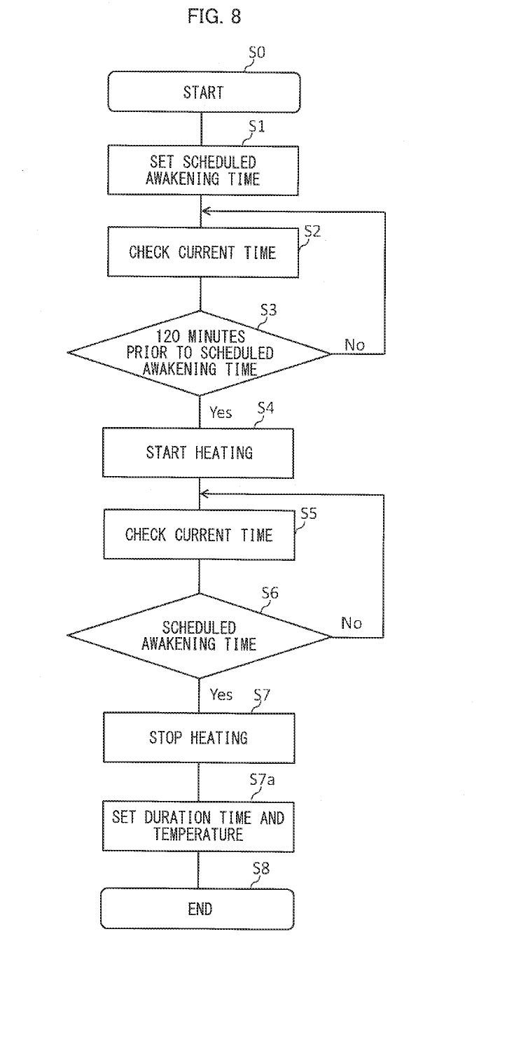

FIG. 6 is a perspective view schematically illustrating the sleep control system 1a used in Embodiment 2. FIG. 7 is a block diagram illustrating a configuration of a control section 3a used in Embodiment 2. FIG. 8 is a flowchart showing an operation of the sleep control system 1a in accordance with Embodiment 2.

As illustrated in FIG. 6, the sleep control system 1a of Embodiment 2 is provided with the vibration sensor 10 in a heating section 2a. The vibration sensor 10 may be a vibration sensor including a piezoelectric element, an ultrasonic Doppler sensor, or the like. The vibration sensor 10 is used to detect vibration caused by, for example, turning over of a human body.

As illustrated in FIG. 7, the control section 3a of Embodiment 2 includes therein a control circuit 5a. The control circuit 5a analyzes a total arousal time during sleep, on the basis of the information from the vibration sensor 10, and sets a heating duration time and a heating temperature for carrying out heating by the heating section 2a. Note that the information from the vibration sensor 10 is stored in a memory (not illustrated) in the control circuit 5a.

As shown in FIG. 8, the sleep control system 1a of Embodiment 2 is arranged to have Step S7a after the end of heating by the heating section 2 in Step S7 and before the end of an operation of the sleep control system 1a in Step S8.

In Step S7a, the control circuit 5a sets a heating duration time and a heating temperature for carrying out heating by the heating section 2a, on the basis of information from the vibration sensor 10. Then, when the sleep control system 1a is caused to operate next time, the heating section 2a carries out heating for the heating duration time and at the heating temperature, which heating duration time and heating temperature are set after current heating is stopped.

Then, the control circuit 5a shortens the heating duration time of heating carried out by the heating section 2a, and/or decreases the heating temperature in a case where the total arousal time during sleep is longer than a reference that is 5% of a total sleep time.

For example, in a case where the heating section 2a carries out heating currently at 40° C. from 120 minutes prior to a scheduled awakening time and a total arousal time during sleep on the basis of information from the vibration sensor 10 is 6% of the total sleep hours, the heating section 2a carries out heating next time at 39° C. from 120 minutes prior to the scheduled awakening time.

As a result, in the sleep control system 1a of Embodiment 2, the temperature is adjusted depending on sensitivity of each individual to temperature. This makes it possible to reliably lead to a pleasant awakening state while causing no disturbance in sleep.

Note that though in the sleep control system 1a of Embodiment 2, the vibration sensor 10 is provided within the heating section 2a, an embodiment of the present invention is not limited to this configuration. The vibration sensor 10 may be provided outside the heating section 2a.

Furthermore, the sleep control system 1a of Embodiment 2 sets, in Step S7a, a heating duration time and a heating temperature for carrying out heating by the heating section 2a, after the heating section 2a stops heating in Step S7. However, an embodiment of the present invention is not limited to this configuration. Such a step of setting the heating duration time and the heating temperature may be provided prior to Step S7, or alternatively, Steps S7 and S7a may be carried out simultaneously.

In addition, the sleep control system 1a of Embodiment 2 sets a heating duration time and a heating temperature for carrying out heating by the heating section 2a in Step S7a, and next heating is carried out for the heating duration time and at the heating temperature which heating duration time and heating temperature are thus set in Step S7a. However, an embodiment of the present invention is not limited to this configuration. Step S7a may be provided before the control circuit 5a determines whether or not the current time is a scheduled awakening time in Step S6, and heating during a current operation may be carried out for the heating duration time an at the heating temperature.

### Embodiment 3

The following discusses a sleep control system 1b in accordance with Embodiment 3 with reference to drawings. Embodiment 3 is different from Embodiment 1 in that the sleep control system 1b is provided with an audio device 11. The description of Embodiment 3 puts the focus on parts different from Embodiment 1.

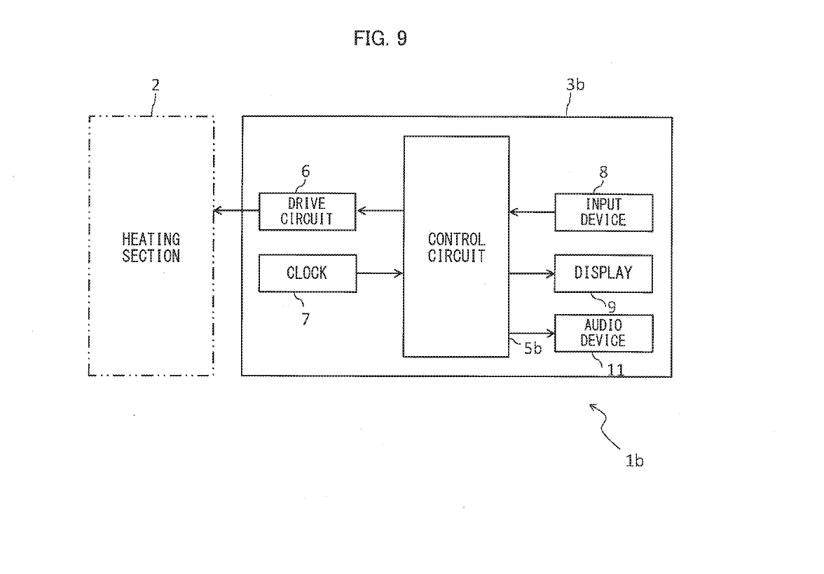

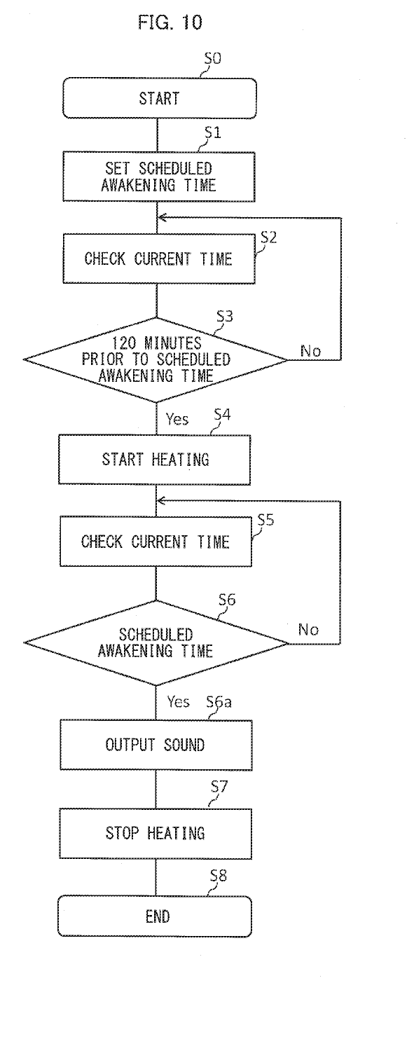

FIG. 9 is a block diagram illustrating a configuration of a control section 3b used in Embodiment 3. FIG. 10 is a flowchart showing an operation of the sleep control system 1b in accordance used in Embodiment 3.

As illustrated in FIG. 9, the sleep control system 1b of Embodiment 3 is provided with an audio device 11 in the control section 3b. Then, the control circuit 5b in the control section 3b generates a signal at a scheduled awakening time, so that the audio device 11 makes a sound as an alarm at the scheduled awakening time in accordance with the signal.

As shown in FIG. 10, in the sleep control system 1b of Embodiment 3, Step S6a is provided after Step S6 in which the control circuit 5b determines whether or not the current time is the scheduled awakening time and before Step S7 in which the heating section 2 stops a heating operation.

In Step S6a, the audio device 11 outputs a sound as an alarm at a scheduled awakening time, in accordance with the signal from the control circuit 5b. As a result, the sleep control system 1b of Embodiment 3 makes it possible not only to reliably lead to awakening at the scheduled awakening time but also to lead to a pleasant awakening state.

Note that though in the sleep control system 1b of Embodiment 3, a heating duration time and a heating temperature for carrying out heating by the heating section 2a is set in Step S6a before the heating section 2a stops heating in Step 7, an embodiment of the present invention is not limited to this configuration. Steps S7 and S6a may be carried out simultaneously.

### Embodiment 4

The following discusses a sleep control system 1 in accordance with Embodiment 4, with reference to a drawing. Embodiment 4 is different from Embodiment 1 in that a heating section 2 does not carry out heating in a period of 0 minute to 30 minutes prior to a scheduled awakening time until the scheduled awakening time. The description of Embodiment 4 puts the focus on parts different from Embodiment 1.

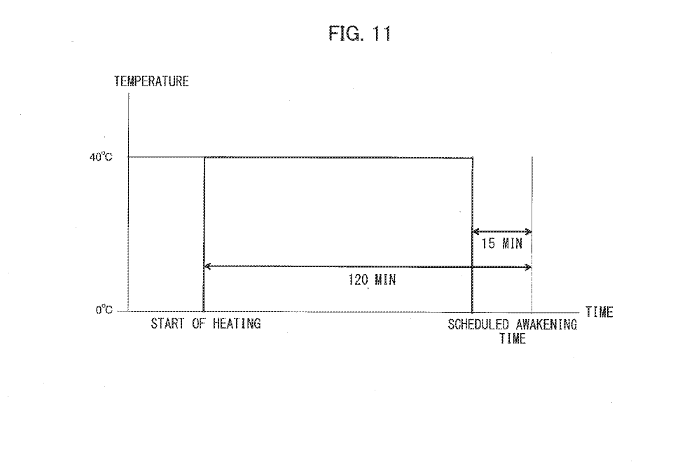

FIG. 11 is a chart showing a change in heating temperature of the heating section 2 used in Embodiment 4. FIG. 11 shows a change in heating temperature of the heating section 2 from a start of heating until a scheduled awakening time.

As shown in FIG. 11, in the sleep control system 1 of Embodiment 4, the heating section 2 carries out heating at a heating temperature of 40° C. from 120 minutes prior to a scheduled awakening time until 15 minutes prior to the scheduled awakening time. Unlike in Embodiment 1, the heating section 2 does not carry out heating in a period from 15 minutes prior to the scheduled awakening time until the scheduled awakening time

In Embodiment 4, the heating section 2 carries out heating from 120 minutes prior to a scheduled awakening time, so that a body temperature at the time of awakening is increased and an arousal level after awakening is accordingly increased. In this point, Embodiment 4 is similar to Embodiment 1. However, since heating by the heating section 2 is easily sensed at the time when the depth of sleep becomes shallow, such as at the time of awakening, heating at such a time of shallow sleep may cause (i) discomfort due to feeling of hotness depending on a season and (ii) feeling of lack of sleep due to increase in the number of arousals during sleep. On this account, Embodiment 4 causes the heating section 2 to stop heating 15 minutes prior to a scheduled awakening time, so as to prevent the above discomfort and feeling of lack of sleep.

Meanwhile, a period of REM sleep may be exhibited in a period from 15 minutes prior to a scheduled awakening time until the scheduled awakening time. Even in this case, since cycles of exhibition of REM sleep are 90-minute cycles, heating is carried out in a last period of REM sleep prior to the period of REM sleep that is exhibited in the period from 15 minutes prior to the scheduled awakening time until the scheduled awakening time. As a result, Embodiment 4 is considered to still provide an effect that an arousal level after awakening is increased.

Further, in Embodiment 4, the heating section 2 is caused to stop heating 15 minutes prior to a scheduled awakening time. This is intended to prevent discomfort and feeling of lack of sleep at the time when the depth of sleep becomes shallow. However, an embodiment of the present invention is not limited to this configuration. The heating section 2 may be arranged to stop heating 0 minute to 30 minutes prior to a scheduled awakening time.

### Embodiment 5

The following discusses a sleep control system 1 in accordance with Embodiment 5, with reference to a drawing. Embodiment 5 is different from Embodiment 1 in that Embodiment 5 causes a heating section 2 to carry out heating at a heating temperature of lower than 36° C. in a period of 0 minute to 30 minutes prior to a scheduled awakening time until the scheduled awakening time. The description of Embodiment 5 puts the focus on parts different from Embodiment 1.

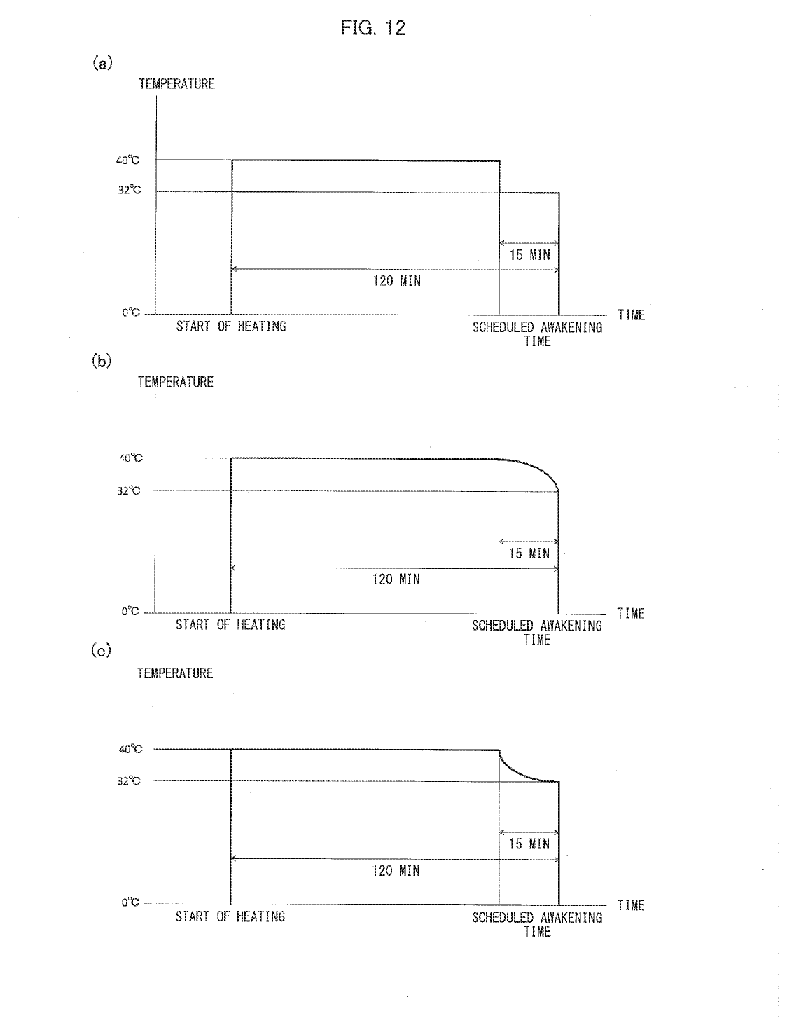

FIG. 12 is a chart showing a change in heating temperature of the heating section 2 used in Embodiment 5. FIG. 12 shows a change in heating temperature of the heating section 2 in a period from a start of heating until a scheduled awakening time. (a) of FIG. 12 shows a case where the heating section 2 carries out heating at a constant heating temperature of 32° C. in a period from 15 minutes prior to a scheduled awakening time until the scheduled awakening time. (b) of FIG. 12 shows a case where the heating section 2 carries out heating at a heating temperature that is decreased from 40° C. to 32° C. at a temperature decrease rate that increases over time, in a period from 15 minutes prior to a scheduled awakening time until the scheduled awakening time. (c) of FIG. 12 shows a case where the heating section 2 carries out heating at a heating temperature that is decreased from 40° C. to 32° C. at a temperature decrease rate that decreases over time, in a period from 15 minutes prior to a scheduled awakening time until the scheduled awakening time.

As shown in FIG. 12, the heating section 2 carries out heating at a heating temperature of 40° C. in a period from 120 minutes prior to a scheduled awakening time until 15 minutes prior to the scheduled awakening time. In this point, Embodiment 5 is similar to Embodiment 1. However, unlike Embodiment 1, in (a) of FIG. 12, the heating section 2 carries out heating at a constant heating temperature of 32° C. in a period from 15 minutes prior to a scheduled awakening time until the scheduled awakening time. Meanwhile, unlike Embodiment 1, in (b) of FIG. 12, the heating section 2 carries out heating at a heating temperature that is decreased from 40° C. to 32° C. at a temperature decrease rate that increases over time, in a period from 15 minutes prior to a scheduled awakening time until the scheduled awakening time. Further, unlike Embodiment 1, in (c) of FIG. 12, the heating section 2 carries out heating at a heating temperature that is decreased from 40° C. to 32° C. at a temperature decrease rate that decreases over time, in a period from 15 minutes prior to a scheduled awakening time until the scheduled awakening time.

In Embodiment 5, the heating section 2 carries out heating from 120 minutes prior to a scheduled awakening time, so that a body temperature at the time of awakening is increased and an arousal level after awakening is accordingly increased. In this point, Embodiment 5 is similar to Embodiment 1. Since heating by the heating section 2 is easily sensed at the time when the depth of sleep becomes shallow, such as at the time of awakening, heating at such a time of shallow sleep may cause (i) discomfort due to feeling of hotness depending on a season and (ii) feeling of lack of sleep due to increase in the number of arousals during sleep.

On this account, Embodiment 5 decreases the heating temperature of the heating section 2 from 15 minutes prior to a scheduled awakening time, so as to prevent the above discomfort and feeling of lack of sleep. By setting the heating temperature of the heating section 2 at 32° C. at the scheduled awakening time, the heating temperature becomes lower than an average human body temperature. This makes it possible to prevent discomfort and feeling of lack of sleep. Further, in a configuration in which heating by the heating section 2 is not stopped but the heating section 2 carries out heating at the heating temperature of 32° C., it is possible to more easily yield an effect that an arousal level after awakening is increased. This is because the configuration prevents a drastic decrease of a body temperature and at the same time, prevents impairment of a pleasant awakening effect that is to be obtained by heating from 120 minutes prior to a scheduled awakening time.

Meanwhile, a period of REM sleep may be exhibited in a period from 15 minutes prior to a scheduled awakening time until the scheduled awakening time. Even in this case, since cycles of exhibition of REM sleep are 90-minute cycles, heating is carried out in a last period of REM sleep prior to the period of REM sleep that is exhibited in the period from 15 minutes prior to the scheduled awakening time until the scheduled awakening time. As a result, Embodiment 5 is considered to still provide an effect that an arousal level after awakening is increased.

Furthermore, in Embodiment 5, the heating temperature of the heating section 2 is decreased from 15 minutes prior to a scheduled awakening time, so that discomfort and feeling of lack of sleep are prevented at the time when the depth of sleep becomes shallow. However, an embodiment of the present invention is not limited to this configuration. The heating temperature of the heating section 2 may be arranged to be decreased from 0 minute to 30 minutes prior to a scheduled awakening time.

In addition, in Embodiment 5, the heating temperature is set at a temperature lower than 36° C., in consideration of an average human body temperature. Accordingly, at a scheduled awakening time, the heating temperature of the heating section 2 is decreased to 32° C. However, an embodiment of the present invention is not limited to this configuration. The heating temperature may be set at any temperature in a range of not lower than 31° C. and lower than 36° C., in consideration of keeping comfortable warmth at a temperature lower than an average human body temperature.

### Embodiment 6

The following discusses a sleep control system 1 in accordance with Embodiment 6, with reference to a drawing. Embodiment 6 is different from Embodiment 1 in that Embodiment 6 causes a heating section 2 to carry out heating such that a heating temperature is in a range of 36° C. to 41° C. at a time point in a range of 90 minutes to 150 minutes prior to a scheduled awakening time and then gradually decreased over time from the time point so as to reach a heating temperature lower than 36° C. at the scheduled awakening time. The description of Embodiment 6 puts the focus on parts different from Embodiment 1.

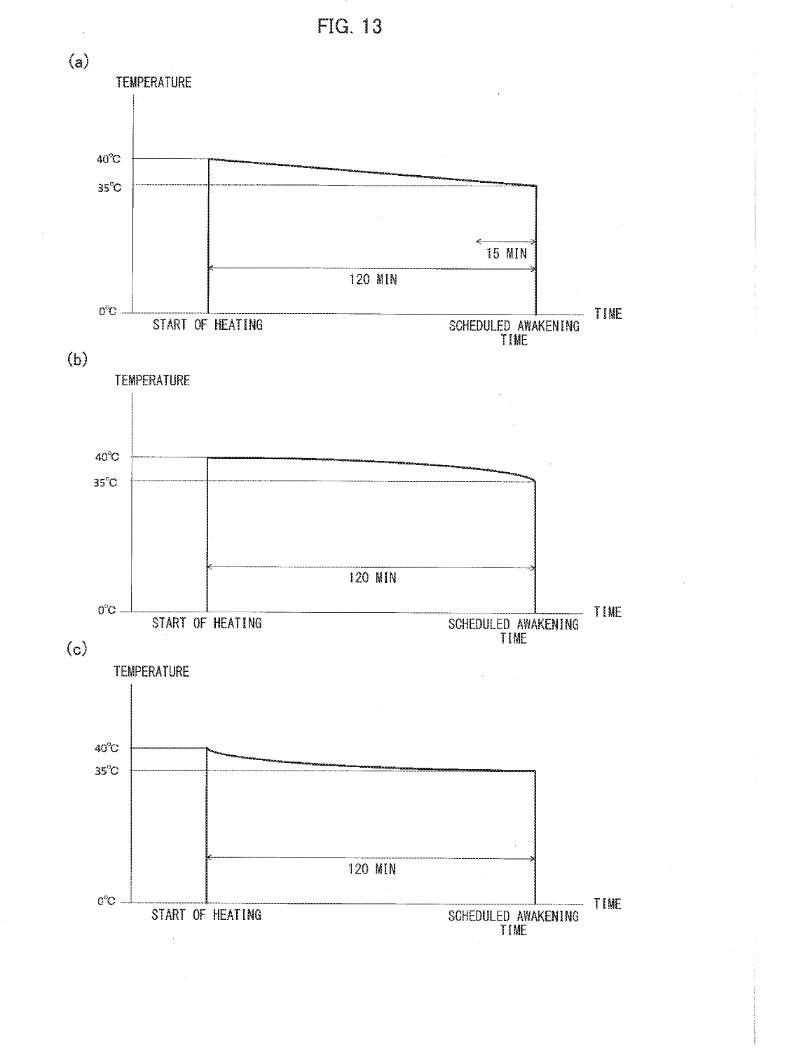

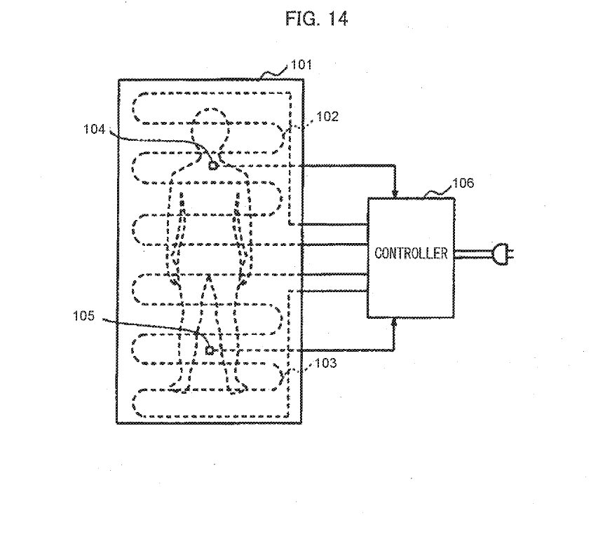

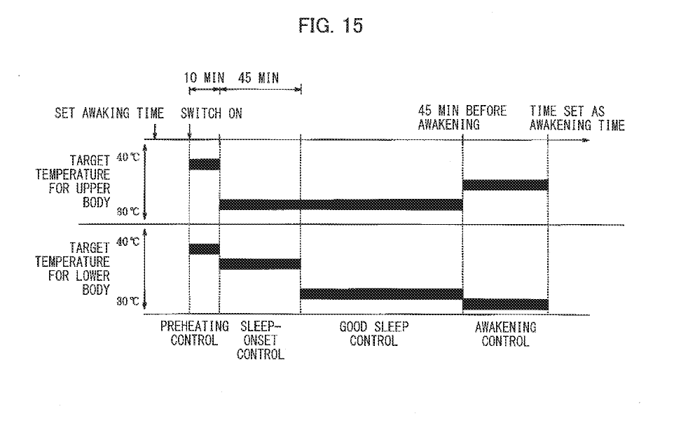

FIG. 13 is a chart showing a change in heating temperature of the heating section 2 used in Embodiment 6. FIG. 13 shows a change in heating temperature of the heating section 2 in a period from a start of heating to a scheduled awakening time. (a) of FIG. 13 shows a case where the heating section 2 carries out heating at a heating temperature that is decreased from 40° C. to 35° C. at a constant temperature decrease rate over time, in a period from 120 minutes prior to a scheduled awakening time until the scheduled awakening time. (b) of FIG. 13 shows a case where the heating section 2 carries out heating at a heating temperature that is decreased from 40° C. to 35° C. at a temperature decrease rate that increases over time, in a period from 120 minutes prior to a scheduled awakening time until the scheduled awakening time. (c) of FIG. 13 shows a case where the heating section 2 carries out heating at a heating temperature that is decreased from 40° C. to 35° C. at a temperature decrease rate that decreases over time, in a period from 120 minutes prior to a scheduled awakening time until the scheduled awakening time.

Unlike in Embodiment 1, as shown in FIG. 13, the heating section 2 carries out heating such that a heating temperature is set at 40° C. at a time point 120 minutes prior to a scheduled awakening time and then gradually decreased over time from the time point so as to reach a heating temperature of 35° C. at the scheduled awakening time.

In Embodiment 6, the heating section 2 carries out heating from 120 minutes prior to a scheduled awakening time, so that a body temperature at the time of awakening is increased and an arousal level after awakening is accordingly increased. In this point, Embodiment 6 is similar to Embodiment 1. Meanwhile, in Embodiment 6, though the heating temperature is gradually decreased from 120 minutes prior to the scheduled awakening time, the heating temperature is set at a temperature not lower than 36° C. in a period of 0 minute to 30 minutes prior to the scheduled awakening time. Accordingly, Embodiment 6 is considered to yield an effect that an arousal level after awakening is increased. Since heating by the heating section 2 is easily sensed at the time when the depth of sleep becomes shallow, such as at the time of awakening, heating at such a time of shallow sleep may cause feeling of hotness depending on a season. This may result in discomfort, feeling of lack of sleep, and an increase in the number of arousals during sleep.

On this account, Embodiment 6 gradually decreases the heating temperature of the heating section 2 from 120 minutes prior to a scheduled awakening time, so as to have a lower heating temperature of the heating section 2 at the scheduled awakening time. Embodiment 6 thereby prevents the above discomfort and feeling of lack of sleep. By setting the heating temperature of the heating section 2 at 35° C. at the scheduled awakening time, the heating temperature becomes lower than an average human body temperature. This makes it possible to prevent discomfort and feeling of lack of sleep. Further, in a configuration in which heating by the heating section 2 is not stopped but the heating section 2 carries out heating at the heating temperature of 35° C., it is possible to more easily yield an effect that an arousal level after awakening is increased. This is because the configuration prevents a drastic decrease of a body temperature and at the same time, prevents impairment of a pleasant awakening effect that is to be obtained by heating from 120 minutes prior to a scheduled awakening time.

Meanwhile, in Embodiment 6, the heating temperature is set at a temperature lower than 36° C., in consideration of an average human body temperature. Accordingly, at a scheduled awakening time, the heating temperature of the heating section 2 is decreased to 35° C. However, an embodiment of the present invention is not limited to this configuration. The heating temperature may be set at any temperature in a range of not lower than 31° C. and lower than 36° C., in consideration of keeping comfortable warmth at a temperature lower than an average human body temperature.

Though the above has specifically described Embodiments 1 to 6, the present invention is not limited to the above-described embodiments. An embodiment derived from a proper combination of technical means each disclosed in any of the above six embodiments is also encompassed in the technical scope of the present invention.

### INDUSTRIAL APPLICABILITY

A sleep control system in accordance with an embodiment of the present invention can be widely applied to general sleep control systems for leading to pleasant awakening.

### REFERENCE SIGNS LIST

1 sleep control system
2 heating section
3 control section
4 mattress
5 control circuit
6 drive circuit
7 clock
8 input device
9 display
10 vibration sensor
11 audio device

1. A sleep control system comprising:
a heating section being provided in contact with a human body and heating the human body; and
a control section for controlling a heating operation carried out by the heating section,
the heating section carrying out heating at a temperature in a range of 36° C. to 41° C. in a period from time for starting heating until a scheduled awakening time, the time for staring heating being in a period of 90 minutes to 150 minutes prior to the scheduled awakening time, and
the heating section carrying out no heating at a temperature higher than 33° C. in a period from bedtime until the time for starting heating or in a period after elapse of a given time from the bedtime and until the time for starting heating.

2. A sleep control system comprising:
a heating section being provided in contact with a human body and heating the human body; and
a control section for controlling a heating operation carried out by the heating section,
the heating section not only carrying out heating at a temperature in a range of 36° C. to 41° C. in a period from time for starting heating until 0 minute to 30 minutes prior to a scheduled awakening time, but also carrying out heating at a temperature in a range of not lower than 31° C. and lower than 36° C. in a period of 0 minute to 30 minutes prior to the scheduled awakening time, the time for starting heating being in a period of 90 minutes to 150 minutes prior to the scheduled awakening time, and
the heating section carrying out no heating at a temperature higher than 33° C. in a period from bedtime until the time for starting heating or in a period after elapse of a given time from the bedtime and until the time for starting heating.

3. The sleep control system as set forth in claim 1, wherein the heating section carries out no heating in the period from bedtime until the time for starting heating or in a period after elapse of a given time from the bedtime and until the time for starting heating.

4. The sleep control system as set forth in claim 2, wherein the heating section is provided below the human body.

5. The sleep control system as set forth in claim 3, wherein the heating section is formed so as to have a size corresponding to an upper body from shoulder tips to buttocks of the human body.

6. The sleep control system as set forth in claim 1, further comprising:
a vibration sensor for detecting vibration of the human body,
the control section setting a heating duration time and a heating temperature for carrying out heating by the heating section on the basis of information from the vibration sensor.

7. The sleep control system as set forth in claim 1, further comprising:
an audio device for outputting a sound,
the control section causing a sound to be outputted from the audio device at the scheduled awakening time, the sound serving as an alarm for the scheduled awakening time.
